(19) United States
(12) Patent Application Publication
(10) Pub. No.: US 2026/0192009 A1
(43) Pub. Date: Jul. 9, 2026

(54) APPARATUS FOR AROMA DISPENSING

(71) Applicant: Yisroel Meir Konikov, Gush Etzion (IL)

(72) Inventor: Yisroel Meir Konikov, Gush Etzion (IL)

(21) Appl. No.: 19/014,163

(22) Filed: Jan. 8, 2025

(51) Int. Cl. A61L 9/12; A61L 9/012

(52) U.S. Cl. CPC A61L 9/12; A61L 9/012; A61L 2209/13

(57) ABSTRACT

Disclosed is an apparatus for aroma dispensing. The apparatus includes at least one storage container with a sticker on at least one side, allowing it to stick to a consumer product packaging. The storage container is designed to accommodate at least one aromatic element containing the aroma of a consumer product within the consumer product packaging. Additionally, the storage container features at least one tab that can be lifted to enable the evaporation of the consumer product's aroma from the aromatic element, all without opening the packaging. Furthermore, the apparatus ensures convenient and controlled aroma release, enhancing the overall consumer experience.

### FIELD OF THE INVENTION

The present disclosure relates to an aroma dispensing apparatus. Moreover, the present disclosure relates to an apparatus for dispensing the aroma of a consumer product.

### BACKGROUND

Advancements in the olfactory technology domain have gained popularity over the years due to a plethora of applications such as enhancing consumer product evaluation and decision-making processes. Olfactory technology, also known as scent technology, focuses on capturing and reproducing scents to provide users with a realistic olfactory experience. This technology has found applications in various industries, including food and beverage, cosmetics, cleaning products, and personal care. By allowing users to smell a consumer product before purchasing it, olfactory technology enables them to assess the scent and make informed decisions about the product's suitability for their preferences and needs. This advancement has the potential to revolutionize the way consumers interact with products, providing a more immersive and sensory shopping experience.

However, the current state of the olfactory technology domain presents certain challenges and limitations. One of the primary problems is the inability to accurately capture and reproduce complex scents with high fidelity. The intricate composition of many consumer products, such as perfumes or spices, poses a significant challenge in replicating their exact scent profiles. Additionally, the existing methods for delivering scents to users often lack precision and control, resulting in a less authentic olfactory experience. Furthermore, the cost and complexity associated with implementing olfactory technology in consumer products and retail environments have hindered its widespread adoption. Despite these challenges, researchers and inventors have made significant strides in developing innovative solutions to overcome these limitations and improve the overall olfactory experience for consumers.

Therefore, in the light of the foregoing discussion, there exists a need to overcome the aforementioned drawbacks.

### SUMMARY

The aim of the present disclosure is to provide an apparatus for aroma dispensing to enhance the olfactory experience of consumer products by conveniently releasing their aromas without opening the packaging.

Another aim of the present disclosure is to provide an apparatus for aroma dispensing to address the challenge of effectively releasing the aroma of consumer products without compromising the integrity of the packaging.

Another aim of the present disclosure is to provide an apparatus that allows a user to experience the smell of a consumer product before buying the consumer product.

Advantageous features and additional implementations are set out in the appended dependent claims.

Embodiments of the present disclosure substantially eliminate or at least partially address the aforementioned problems in the prior art and improve the convenience and efficiency of aroma dispensing by allowing the aroma of consumer products to be released without the need to open the packaging of the consumer product, thereby preserving the freshness and integrity of the product, and allowing the user to make a better buying decision.

In an aspect, embodiments of the present disclosure comprise an apparatus for aroma dispensing, wherein the apparatus comprises:
at least one storage container comprising a sticker on at least one side of the storage container, wherein the sticker is configured to stick the storage container to at least one consumer product packaging,
wherein the storage container is configured to accommodate at least one aromatic element comprising an aroma of consumer product inside the consumer product packaging,
wherein the storage container comprises at least one tab, wherein the tab is configured to be lifted to allow the aroma of the consumer product to evaporate from the aromatic element without opening the consumer product packaging.

Optionally, the aromatic element is a high viscosity gel, and wherein the high viscosity gel stores the aroma of the consumer product inside the high viscosity gel. The high viscosity gel may have a viscosity between 100,000 cps and 100,000,000 cps, inclusive.

Optionally, the aromatic element is the consumer product itself.

Optionally, the sticker is configured to stick the storage container on an outside surface of the consumer product packaging, and wherein the storage container allows a user to smell the consumer product before buying or using the consumer product.

Embodiments of the present disclosure substantially eliminate or at least partially address the aforementioned problems in the prior art and improve the user's buying experience.

Additional aspects, advantages, features, and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

A better understanding of the present invention may be obtained through the following examples which are set forth to illustrate but are not to be construed as limiting the present invention.

Throughout the description and claims of this specification, the words “comprise”, “include”, “have”, and “contain” and variations of these words, for example “comprising” and “comprises”, mean “including but not limited to”, and do not exclude other components, items, integers, or steps not explicitly disclosed also to be present. Moreover, the singular encompasses the plural unless the context requires otherwise requires. In particular, where the indefinite article is used, the specification is to be understood as contemplating plurality as well as singularity, unless the context requires otherwise.

### BRIEF DESCRIPTION OF THE DRAWINGS

FIG. 1 is an illustration of an apparatus for aroma dispensing that is stuck outside to the consumer product packaging.

FIG. 2 is an illustration of the apparatus for aroma dispensing that is stuck outside to the consumer product packaging, and having a tab.

### DETAILED DESCRIPTION OF EMBODIMENTS

The following detailed description illustrates embodiments of the present disclosure and the ways in which they can be implemented. Although some modes of carrying out the present disclosure have been disclosed, those skilled in the art would recognize that other embodiments for carrying out or practicing the present disclosure are also possible.

The terms “a” and “an” herein do not denote a limitation of quantity, but rather denote the presence of at least one of the referenced items. The terms “having”, “comprising”, “including”, and variations thereof signify the presence of a component.

In a first aspect, the present disclosure provides an apparatus for aroma dispensing wherein:
at least one storage container comprises a sticker on at least one side of the storage container, wherein the sticker is configured to stick the storage container to at least one consumer product packaging;
the storage container is configured to accommodate at least one aromatic element comprising an aroma of a consumer product configured inside the consumer product packaging; and
the storage container comprises at least one tab, wherein the tab is configured to be lifted to allow the aroma of the consumer product to evaporate from the aromatic element without opening the consumer product packaging.
The present disclosure provides an innovative solution in the form of a storage container that can be attached to consumer product packaging, allowing potential customers to experience the scent/smell of the product before making a purchase.

The storage container incorporates a sticker that securely holds the aroma of the consumer product, ensuring its preservation until it is intentionally released. This is achieved through the use of a specialized material, such as a hard material or gel, which effectively seals the smell of the consumer product in the storage compartment. The storage container comprises a tab that can be lifted, enabling the aroma to evaporate and be smelled without the need to open the consumer product packaging.

Additionally, the storage container having the sticker offers alternative methods of scent release, such as a tab that automatically closes or a button that releases a small amount of smell of the consumer product.

These features work synergistically to provide several advantages. Firstly, customers can make more informed purchasing decisions by experiencing the scent of the consumer product beforehand. This can enhance customer satisfaction and reduce the likelihood of returns or dissatisfaction with the product's smell. Secondly, the sealed storage compartment ensures that the aroma remains intact until intentionally released, preventing any accidental or premature scent exposure. Lastly, the various scent release options cater to different preferences, allowing customers to choose their preferred method of experiencing the scent. Overall, this inventive sticker provides a convenient and reliable way for customers to sniff and evaluate the scent of a product, enhancing the overall shopping experience.

The terms “consumer product” and “product” have been used interchangeably throughout the present disclosure.

Throughout the present disclosure, the term “storage container” refers to a receptacle designed to hold and protect items, typically made of durable materials such as plastic or metal. The term “sticker” refers to a small piece of adhesive material, often with printed information or graphics, that can be attached (i.e. “stuck”) to various surfaces for identification or decoration purposes. The term “consumer product packaging” refers to the materials and design used to enclose and present a product to the end user, including boxes, bags, or other forms of containers.

An apparatus for aroma dispensing may comprise at least one storage container with a sticker affixed to at least one side of the storage container, wherein the sticker is designed to adhere (i.e. “stick”) to the consumer product packaging. The apparatus for aroma dispensing comprises at least one storage container with a sticker affixed to at least one side. The sticker is designed to stick securely to a consumer product packaging, allowing the storage container to be securely attached. The storage container includes a tab that can be lifted to expose the scent stored inside. The scent is preserved within a material, which can be a hard material or a gel, ensuring that the smell remains sealed until intentionally opened. The tab of the storage container can be opened and closed automatically, or a button can be pressed to release a small amount of scent. The purpose of this apparatus is to provide a means for consumers to experience the aroma of a product before making a purchase. By attaching the storage container to the product packaging, the storage container allows for easy access to the scent. The ability to lift the tab or press a button provides a convenient way for consumers to release and smell the preserved scent. The apparatus enables consumers to evaluate the aroma of a product before purchasing, enhancing the overall shopping experience. The powerful adhesive properties of the sticker ensure that the storage container remains securely attached to the product packaging. The sealed storage compartment effectively preserves the scent until it is intentionally opened, maintaining the quality and longevity of the aroma. The option to open the tab or press a button provides flexibility in how the scent is released, catering to individual preferences.

The storage container with the sticker is removably stuck to the consumer product packaging. The storage container with the sticker can be completely removed from the consumer product packaging. This allows a consumer to smell the aroma of a product without having to lift the entire consumer product packaging, which is beneficial for consumer products with corresponding consumer product packaging that are quite heavy. Furthermore, the storage containers and/or stickers may be color-coded to prevent mix-ups when the stickers are removed from their corresponding consumer product packaging.

Throughout the present disclosure, the term “aromatic element” refers to a chemical compound or substance that possesses a distinct and often pleasant odour, often used in the formulation of perfumes, fragrances, and other scented products. The term “consumer product” refers to any tangible item or good that is intended for use or consumption by individuals for personal or household purposes, including but not limited to cosmetics, cleaning agents, food and beverages, and personal care items. In the apparatus for aroma dispensing, the storage container is configured to accommodate at least one aromatic element that comprises the aroma of a consumer product inside the packaging of the consumer product. The apparatus for aroma dispensing is achieved through the use of a sticker that can be securely adhered to a consumer product packaging. The sticker includes a tab that can be lifted, allowing the scent to evaporate and be smelled. Furthermore, the tab can be a made up of a rigid material which allows the tab to return its original position.

The smell is stored inside the sticker using a material, which can be either a hard material or a gel, that effectively preserves the scent. The storage compartment for the smell remains sealed until it is opened for softening and then closed again. The smell can be accessed by either opening a tab with automatic closing or by pressing a button that releases a small amount of scent. The purpose of this apparatus is to provide consumers with the ability to experience the aroma of a product before making a purchase. By incorporating the scent directly into the packaging, potential buyers can have a more immersive and sensory shopping experience. This can help them make more informed decisions and enhance their overall satisfaction with the product. The technical effect of this apparatus is that it allows for controlled aroma dispensing from the consumer product packaging. The storage container, in the form of a sticker, securely holds the aromatic element and prevents any leakage or premature evaporation of the scent. The sealed storage compartment ensures that the scent remains intact until it is intentionally released by the user. The option to either open a tab or press a button provides flexibility in accessing the aroma, catering to different user preferences. Overall, this apparatus enhances the olfactory experience associated with consumer products, offering a unique and engaging way for customers to interact with the product before purchase.

Throughout the present disclosure, the term “tab” refers to a small, flat, and typically rectangular object that is used for various purposes, such as organizing information or providing a means of interaction in user interfaces. Furthermore, the tab can be a made up of a rigid material which allows the tab to return its original position. The term “aroma” refers to the characteristic smell or fragrance emitted by a substance, which can be perceived by the olfactory system. The term “evaporate” refers to the process by which a liquid or solid substance changes into a gaseous state, typically due to the application of heat or the reduction of pressure. The apparatus for aroma dispensing may comprise a storage container with at least one tab, wherein the tab is designed to be lifted, enabling the aroma of the consumer product to evaporate from the aromatic element without the need to open the packaging of the consumer product. Furthermore, the tab can be a made up of a rigid material which allows the tab to return its original position. The tab can be made of but not limited polyvinyl acetate, polyvinyl chloride, Bakelite®, or a combination thereof. The apparatus for aroma dispensing comprises a storage container with at least one tab. The tab is designed to be lifted, allowing the aroma of the consumer product to evaporate from the aromatic element. This is done to enable potential customers to smell the product before purchasing it, providing them with a sensory experience and helping them make an informed decision. The storage container with the lift tab preserves the scent of the consumer product using a material that seals the smell storage compartment. This ensures that the scent remains intact until the tab is lifted, allowing the aroma to be released for smelling. The option to either open the tab or press a button to release a small amount of scent provides flexibility and convenience for the user.

In accordance with an embodiment, the aromatic element can be a high viscosity gel. Throughout the present disclosure, the term “high viscosity gel” refers to a gelatinous substance characterized by its resistance to flow, possessing a thick and sticky consistency due to its high molecular weight and strong intermolecular forces. The aromatic element in the apparatus is a high viscosity gel, which is used to store and preserve the scent within the storage container. The gel material acts as a barrier, preventing the scent from escaping until its corresponding storage container intentionally opened or until the scent is otherwise activated like by pressing a button. The use of a high viscosity gel as the aromatic element serves multiple purposes. Firstly, it ensures that the scent is securely contained within the storage container, preventing any accidental leakage or premature evaporation. Secondly, the gel material helps to preserve the scent for an extended period, maintaining its intensity and quality until it is intentionally released. By utilizing a high viscosity gel as the aromatic element, the apparatus provides a reliable and controlled method for storing and releasing scents. The gel's viscosity helps to maintain a sealed environment, preventing any unwanted scent leakage. Additionally, the gel's properties contribute to the longevity and preservation of the scent, ensuring that it remains potent and appealing until it is intentionally activated.

In accordance with an embodiment, the high viscosity gel stores the aroma of the consumer product inside the high viscosity gel. The apparatus utilizes a sticker that adheres the storage container strongly to the consumer product, allowing potential customers to sniff and experience the aroma before making a purchase. The storage container incorporates a lift-up tab that, when raised, triggers the evaporation of the scent for smelling. The purpose of this invention is to provide a convenient and interactive way for consumers to evaluate the scent of a product before buying it. By incorporating a scent storage compartment within the storage container, the aroma can be preserved and released when desired, ensuring that the scent remains intact until it is intentionally opened. The high viscosity gel is employed as the material for storing the aroma within the storage container. This gel effectively preserves the scent, whether it is a hard material or a gel consistency. The sealed storage compartment ensures that the aroma remains contained until it is intentionally opened, either by lifting the tab with automatic closing or by pressing a button to release a controlled amount of scent. This technical arrangement allows for repeated use of the sticker while maintaining the freshness and integrity of the stored aroma.

In accordance with an embodiment, the aromatic element is the consumer product itself. The apparatus utilizes a sticker that is designed to adhere the storage container securely to the consumer product packaging. This storage container includes a tab that can be lifted, allowing the scent of the product to be released and smelled before purchase. The purpose of this design is to provide potential consumers with the ability to experience the aroma of the product before making a purchase decision. By incorporating the scent directly from the consumer product itself, it offers a more authentic and accurate representation of the product's fragrance. The apparatus employs a storage compartment within the sticker, which is sealed to preserve the scent until it is intentionally opened. This compartment utilizes a material, such as a hard material or gel, to effectively retain and protect the aroma. The tab of the compartment can be opened and closed automatically, or a button can be pressed to release a controlled amount of scent. This design ensures that the aromatic element, which is the consumer product itself, remains securely contained until the user chooses to experience the fragrance.

In accordance with an embodiment, the sticker is configured to stick the storage container on an outside surface of the consumer product packaging. The term “outside surface” refers to the external or outermost layer of an object or structure. The sticker is designed to securely adhere to the consumer product packaging, allowing for easy attachment of the storage container. The storage container is integrated into the sticker and is sealed to preserve the scent. The sticker includes a tab that can be lifted to release the scent for sniffing purposes. Additionally, there is an option to open the tab manually, which automatically closes after use, or to press a button that releases a small amount of scent. The purpose of sticking the storage container on the outside surface of the consumer product packaging is to provide a convenient and accessible way for potential customers to experience the scent of the product before making a purchase. By having the storage container integrated into the sticker, it ensures that the scent remains sealed and preserved until it is intentionally opened for sniffing. The technical effect of this configuration is that it allows for controlled and easy access to the scent of the product. The sticker's strong adhesive ensures that the storage container remains securely attached to the packaging, preventing accidental detachment. The option to lift the tab or press a button provides flexibility in how the scent is released, allowing users to experience the fragrance in a controlled manner. Overall, this design enhances the customer's sensory experience and helps in making informed purchasing decisions.

In accordance with an embodiment, the storage container allows a user to smell the consumer product before buying or using the consumer product. Throughout the present disclosure, the term “user” refers to an individual or entity that interacts with a system, device, typically by providing input, receiving output, or engaging in some form of communication or interaction. In the present disclosure the user can be a buyer or potential buyer of the consumer product.

The apparatus comprises a sticker that is designed to adhere the storage container securely to the consumer product packaging. The storage container includes a tab that can be lifted to release the scent for smelling. Inside the sticker, a smell storage compartment is incorporated, which is sealed to preserve the scent. The smell is stored using a material, such as a hard material or gel, that effectively retains the scent. The storage compartment can be opened to soften the scent and then closed again. The purpose of this design is to allow potential consumers to experience the scent of the product before making a purchase. By providing a means to smell the product, users can make more informed decisions and evaluate the scent's appeal, which can be a significant factor in their purchasing decision. The technical effect of this apparatus is that it enables users to directly smell the consumer product without opening the consumer product packaging or coming into direct contact with the product itself. The sealed storage compartment within the sticker ensures that the scent is preserved until it is intentionally released. This design provides a convenient and hygienic way for users to experience the scent of the product, enhancing the overall user experience and potentially influencing their purchasing decision.

### DETAILED DESCRIPTION OF THE DRAWINGS

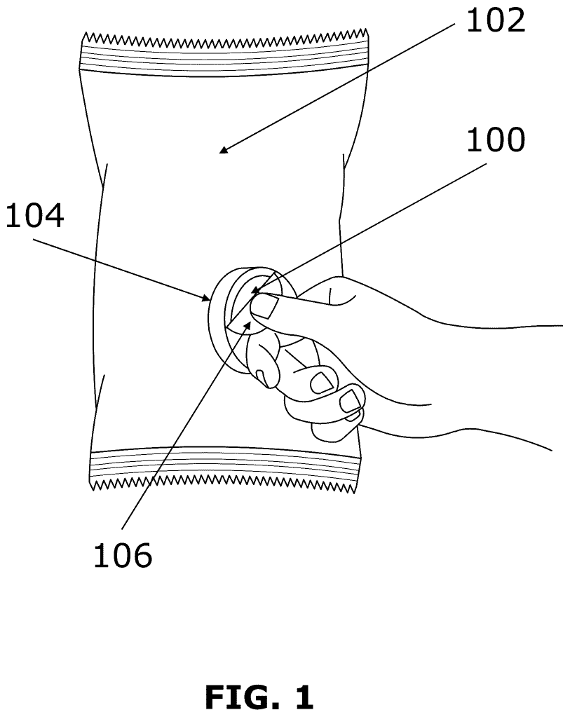

Referring to FIG. 1, illustrated is an apparatus 100 for aroma dispensing that is stuck outside to the consumer product packaging 102, in accordance with an embodiment of the present disclosure. The apparatus 100 comprises at least one storage container 104 comprising a sticker (not shown in FIG. 1) on at least one side of the storage container 104, wherein the sticker is configured to stick the storage container 104 to at least one consumer product packaging 102. The storage container 104 is configured to accommodate at least one aromatic element comprising an aroma of a consumer product configured inside the consumer product packaging 102. The storage container 104 comprises at least one tab 106, wherein the tab 106 is configured to be lifted to allow the aroma of the consumer product to evaporate from the aromatic element without opening the consumer product packaging 102.

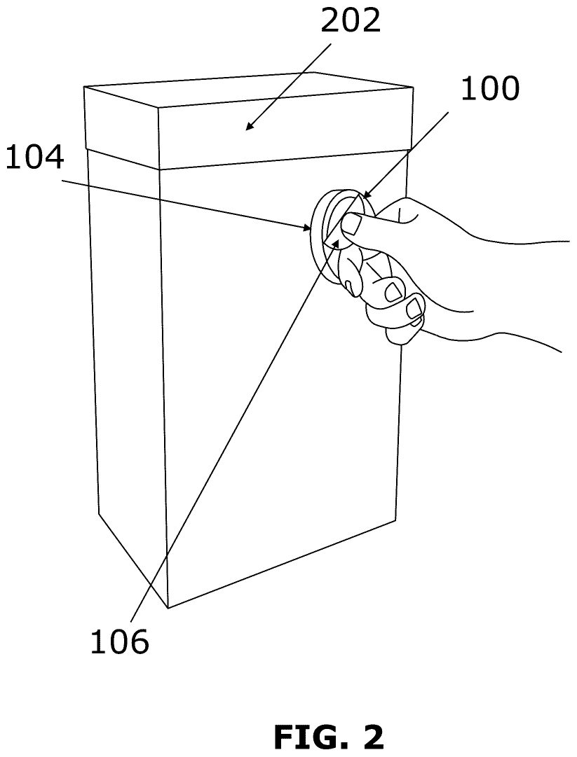

Referring to FIG. 2, illustrated is an apparatus 100 for aroma dispensing that is stuck outside to the consumer product packaging 202, and having a tab 106, in accordance with an embodiment of the present disclosure. The apparatus 100 comprises the storage container 104 comprising a sticker (not shown in FIG. 2) on one side of the storage container 104, wherein the sticker is configured to stick the storage container 104 to the consumer product packaging 202. The storage container 104 is configured to accommodate an aromatic element comprising an aroma of a consumer product inside the consumer product packaging 202. The storage container 104 comprises one tab 106, wherein the tab 106 is configured to be lifted to allow the aroma of the consumer product to evaporate from the aromatic element without opening the consumer product packaging 202.

We claim:

1. An apparatus for aroma dispensing comprising:
at least one storage container comprising a sticker on at least one side of the storage container, wherein the sticker is configured to stick the storage container to at least one consumer product packaging,
wherein the storage container is configured to accommodate at least one aromatic element comprising an aroma of a consumer product configured inside the consumer product packaging,
wherein the storage container comprises at least one tab, wherein the tab is configured to be lifted to allow the aroma of the consumer product to evaporate from the aromatic element without opening the consumer product packaging.

2. The apparatus according to claim 1, wherein the aromatic element is a high viscosity gel, and wherein the high viscosity gel stores the aroma of the consumer product inside the high viscosity gel.

3. The apparatus according to claim 1, wherein the aromatic element is the consumer product itself.

4. The apparatus according to claim 1, wherein the sticker is configured to stick the storage container on an outside surface of the consumer product packaging, and wherein the storage container allows a user to smell the consumer product before buying or using the consumer product.
